SEQUENCE LISTING (4 sequences)

SEQ ID NO: 1 - protein, 10 aa, Artificial Sequence
  Synthetic- nivolumab binding sequence
LDSPDRPWNP

SEQ ID NO: 2 - DNA, 30 bp, Artificial Sequence
  Synthetic- sequence encoding nivolumab binding sequence
ttagactccccagacaggccctggaacccc

SEQ ID NO: 3 - protein, 26 aa, Artificial Sequence
  Synthetic- pembrolizumab binding sequence
NQTDKLAAFPEDRSQPGQDCRFRVTQ

SEQ ID NO: 4 - DNA, 79 bp, Artificial Sequence
  Synthetic- sequence encoding pembrolizumab binding sequence
caaccagacggacaagctggccgccttccccgaggaccgcagccagcccggccaggactgccgcttccgtgtcacacaa

(19) United States
(12) Patent Application Publication
(10) Pub. No.: US 2024/0075145 A1
(43) Pub. Date: Mar. 7, 2024

(54) ELECTROTRANSFER METHODS FOR TREATING TUMORS

(71) Applicant: UNIVERSITY OF SOUTH FLORIDA, Tampa, FL (US)

(72) Inventors: Loree C. HELLER, Tampa, FL (US); Richard HELLER, Tampa, FL (US); Mark Jeffrey JAROSZESKI, Tampa, FL (US)

(21) Appl. No.: 18/262,548

(22) PCT Filed: Jan. 24, 2022
(86) PCT No.: PCT/US2022/013543

§ 371 Date: Jul. 21, 2023

Related U.S. Application Data
(60) Provisional application No. 63/140,365, filed on Jan. 22, 2021.

(51) Int. Cl. A61K 41/00; A61K 38/17; A61K 38/19; A61K 38/20; A61K 38/21; A61K 48/00; A61P 35/00

(52) U.S. Cl. CPC A61K 41/0047; A61K 38/1774; A61K 38/193; A61K 38/195; A61K 38/208; A61K 38/2086; A61K 38/21; A61K 41/0052; A61K 48/0075; A61P 35/00

(57) ABSTRACT

Disclosed herein are electrotransfer (ET) methods for delivering therapeutic agents to tumors. Also disclosed are methods of using the ET methods for differential delivery of an agent to more than one tissue. For example, the ET methods can be used to deliver soluble peptides of PD1 to tumor tissue to block normal PD1-PDL1 binding while separately using the ET methods to deliver PD1 or PDL1 antigen to another tissue, such as skin or muscle, to induce systemic and polyclonal checkpoint inhibitor antibodies.

### CROSS-REFERENCE TO RELATED APPLICATIONS

This application claims the benefit of priority under 35 U.S.C. § 119(e) of U.S. Provisional Application No. 63/140,365, filed Jan. 22, 2021, the contents of which is incorporated herein by reference in its entirety.

### STATEMENT REGARDING FEDERALLY SPONSORED RESEARCH OR DEVELOPMENT

This invention was made with Government Support under Grant No. CA186730 awarded by the National Institutes of Health. The Government has certain rights in the invention.

### SEQUENCE LISTING

A Sequence Listing accompanies this application and is submitted as an ASCII text file of the sequence listing named “173738_02411_ST25.txt” which is 1,434 bytes in size and was created on Jan. 18, 2022. The sequence listing is electronically submitted via EFS-Web with the application and is incorporated herein by reference in its entirety.

### BACKGROUND

The advent of checkpoint inhibitors considerably augmented existing cancer therapies. Checkpoint inhibitors such as PD1 targeting antibodies (e.g. nivolumab, Opdivo, Bristol Myers Squibb, pembrolizumab, Keytruda, Merck) are FDA approved as therapies for many cancers. These inhibitors short-circuit PD1-PDL1 binding, removing interference with T cell function, allowing T cell proliferation, and restoring the anti-tumor activity. They are most effective clinically when combined with surgery, chemotherapy, targeted therapies, and/or radiotherapy (Smyth MJ, et al. Nat Rev Clin Oncol. 2016 13(3):143-58). A major negative of checkpoint inhibitor therapy is the high cost (Andrews A. Am Health Drug Benefits. 2015 8(Spec Issue):9). For example, per Merck, the list price for each Keytruda dose is $9,724.08.

There is a fine line between immunostimulation and immunosuppression, so it is important to deliver a plasmid in a manner to achieve the appropriate expression. When delivering an immunotherapeutic agent, it is critical to control levels of the agent to obtain the correct therapeutic response. When these agents are delivered as a protein, typically they are delivered as a high concentration protein bolus to maintain therapeutic levels which can result in toxic side effects. High doses of immunotherapies can also disrupt the balance between immunostimulation and immunosuppression.

Conversion to a gene therapy has several potential advantages over protein therapy. Gene therapies can be less toxic. Therapeutic protein production by host cells also allows for fewer treatments. Since therapeutic proteins are produced by transfected host cells over a period of time, fewer treatments are necessary. This is less toxic, since it is not necessary to deliver a high concentration protein bolus to maintain therapeutic levels. However, with gene therapies, since the gene is the delivered molecule and the protein is the therapeutic molecule, the pharmacokinetics and pharmacodynamics of the therapeutic dose may not be well defined. This can be circumvented by establishing a more controlled delivery mechanism.

### SUMMARY

In vivo electroporation or electrotransfer (ET) can efficiently deliver nucleic acids to several tissues including solid tumors. While ET in general can deliver nucleic acids to tissues, there is little control over the induced expression levels and distribution within the tissue. Utilizing moderate heat enables more efficient and targeted gene delivery. For example, the agent could be distributed throughout the tissue, could be targeted to specific areas of the tissue, or to specific cells within the tissue.

A second issue with this approach is that there is not a way to verify if successful delivery has been achieved until a therapeutic outcome can be determined. There can be a considerable delay in obtaining this answer, which if negative can result in loss of valuable time when dealing with a therapeutic for a deadly disease. This is a critical aspect of delivering therapeutics particularly for immunotherapy. ET delivery allows the use of impedance monitoring to determine if proper delivery occurs at the time of administering the therapeutic. This increases the reproducibility of the nucleic acid-based medicine.

The advent of checkpoint inhibitors considerably augmented existing cancer therapies. Checkpoint inhibitors such as PD1 targeting antibodies (e.g. nivolumab, Opdivo, Bristol Myers Squibb, pembrolizumab, Keytruda, Merck) are FDA approved as therapies for many cancers. These inhibitors short-circuit PD1-PDL1 binding, removing interference with T cell function, allowing T cell proliferation, and restoring the anti-tumor activity. They are most effective clinically when combined with surgery, chemotherapy, targeted therapies, and/or radiotherapy.

Disclosed herein are plasmids focusing on the extracellular domain or subdomains of PD1. The plasmids encode soluble peptides of PD1, which may bind PDL1 on tumor cells to block normal PD1-PDL1 binding (FIG. 1). To achieve an effective therapeutic response, the plasmid(s) can be delivered directly to the tumor. Combining ET with moderate heat (e.g. about 43° C.) enables the even distribution of the expressed peptide broadly throughout the tumor to assure binding to all available PDL1 targets. Impedance monitoring of the pulse effects on the tissue is incorporated to ensure reproducible delivery. Performing the therapy this way enables tight control of expression, leading to only local immune activation while avoiding the creation of an immunosuppressive tumor microenvironment.

If systemic expression is utilized, these expressed extracellular domain or subdomains act as antigens, inducing systemic and polyclonal checkpoint inhibitor antibodies (FIG. 1). These polyclonal antibodies target the same region as the monoclonal antibodies and should function similarly to monoclonal checkpoint inhibitor antibodies. Native antibodies are produced by the patient, removing the need for repeated recombinant antibody infusions. This could be accomplished by increasing expression within the tumor or by delivery to other tissues such as skin or muscle. For example, the muscle acts as a bioreactor so intramuscular (IM) gene delivery produces high protein levels. IM electroporation of the PD1ex plasmid in mice induces significant, long-term production of anti-PD1 antibody in serum (FIG. 2).

By combining these two therapeutic approaches, this anti-PD1 gene therapy can also work as a dual mechanism therapeutic (FIG. 1). The peptides directly block PD1-PDL1 binding locally within the tumor, while the antibodies block PD1-PDL1 binding systemically. This multipurpose mechanism should allow the proliferation of T cells in the tumor microenvironment while limiting the need for multiple monoclonal antibody injections.

This same dual mechanism concept can be applied to other molecules, including other checkpoint inhibitors using a single checkpoint or combinations of checkpoints. For example, the checkpoint molecules can be PD1, PDL1, CTLA-4, TIM-3, 4.1BB, LAG-3, CD80, CD86, OX40, OX40L, or any combination thereof. In some embodiments, both the first and the second polynucleotides are PD1 or PDL1. In some embodiments, the first polynucleotide encodes PD1 and the second polynucleotide encodes PDL1. For example, a plasmid encoding a PD-1 peptide can be delivered to the tumor, while a plasmid encoding PD-L1 is delivered to muscle or skin to induce the generation of anti-PD-L1 antibodies. The PD-1 peptide will facilitate the local anti-tumor response and initiate an anti-tumor systemic immune response while the anti-PD-L1 antibodies will augment this systemic immune response. This method allows for timing the delivery to induce the antibodies sequentially or simultaneously as needed with respect to the PD-1 peptide anti-tumor effect.

This modified ET approach can efficiently deliver nucleic acids encoding cytokines, chemokines and other immune modulators to produce local or systemic expression. For example, a plasmid encoding a specific cytokine can be delivered directly to the tumor in a specific manner to modify the tumor microenvironment to enhance the anti-tumor effect. Single agents can be delivered. Alternatively, multiple agents can be delivered to act synergistically, thereby augmenting the anti-tumor immune response. Therefore, in some embodiments, one or more of the first or second polynucleotides encode a cytokine and/or chemokine. For example, the cytokine or chemokine can be IL-12, IL-15, GM-CSF, IFNs, CCI19, CCL21, CXCL12, CCL14, CCR7, or any combination thereof.

Controlled delivery of these immunotherapies alone or in combination in a well-defined approach enables the timing of tumor cell death leading to the induction of an adaptive immune response as opposed to an innate response. An adaptive immune response would enable a local therapy to have systemic long-term antitumor effects.

This modified ET approach also enables direct intracellular delivery of impermeable chemotherapeutic agents. This allows a significantly reduced dose, therefore reducing side effects. An effective dose can be directly delivered to the tumor, which concentrates the agent in the tumor or the effective dose can be delivered to the muscle or skin to achieve systemic levels of the agent.

Each of these approaches can be utilized alone or with other cancer therapies such as immunotherapies, surgery, chemotherapy, targeted therapies, and/or radiotherapy. The modified ET approach enables control of the timing of combinations to optimize the anti-tumor effects of each individual therapy.

Disclosed herein is a gene therapy alternative to targeting antibodies. Conversion to a gene therapy has several potential advantages over protein therapy. Since proteins are produced by transfected host cells over a period of time, fewer treatments are necessary. This is less toxic, since it is not necessary to deliver a high concentration protein bolus to maintain therapeutic levels. In addition, host cell production also allows for fewer treatments, which potentially reduces costs.

Non-viral gene therapies can be delivered using electroporation or electrotransfer, which increases cell permeability using tightly controlled electric pulses. Intratumor gene electrotransfer (GET) of a plasmid encoding IL-12 is currently in Phase II clinical trials (Daud A I, et al. J Clin Oncol. 2008 26(36):5896-903; Algazi A, et al. Ann Oncol. 2020 32(4):532-540; Greaney S K, et al. Cancer Immunol Res. 2020 8(2):246-54; Algazi A P, et al. Clin Cancer Res. 2020 26(12):2827-2837) based on a mouse model (Lucas M L, et al. Mol. Ther. 2002 5(6):668-75; Lucas M L, et al. DNA Cell Biol. 2003 22(12):755-63; Heller L, et al. Clin Cancer Res. 2006 12(10):3177-83; Marrero B, et al. Technol. Cancer Res Treat. 2014 13(6):551-60; Shirley S A, et al. Curr Gene Ther. 2015 15(1):32-43; Shi G, et al. Cancers 2018 10(12)). This therapy, ImmunoPulse® IL-12 or tavokinogene telseplasmid electroporation or “Tavo” (oncoSec Medical Inc. San Diego, CA), has been fast-tracked by the FDA for treatment of metastatic melanoma following progression on PD1 blockade.

An anti-PD1 vaccine could be combined with both cutting edge (Tavo) and well-established cancer therapy types. Checkpoint inhibitors are most effective clinically when combined with surgery, chemotherapy, targeted therapies, and/or radiotherapy (Smyth M J, et al. Nat Rev Clin Oncol. 2016 13(3):143-58). In particular, radiotherapy and chemotherapy induced immunogenic cell death, which sensitizes cancers to immunotherapies such as checkpoint inhibitors (Pfirschke C, et al. Immunity 2016 44(2):343-54).

The disclosed compositions and methods provide an alternative to checkpoint antibody therapy that relies on infusing proteins. The method provides for delivering plasmid encoding specific regions of a checkpoint to induce the patient to produce the antibodies thus reducing cost, number of treatments and allows a more personal medicine approach. In addition to inducing antibody production, the antigen expressed can bind to the ligand thus also blocking the interaction between for example PD1 and PDL1. In some embodiments, the amount and location of this expression is carefully controlled by the disclosed modified ET methods.

Cancer immunotherapy is dependent on a robust T-cell response. It was discovered that various checkpoints exist that are exhaustion signals for T-cells. These checkpoints reduce or prevent T-cell responses when bound to their ligand. Checkpoint inhibitor therapy involves infusion of antibodies against one or more checkpoints. This therapy is expensive and has potential side effects. In some embodiments, the disclosed methods utilize plasmid DNA encoding specific antigenic regions of the checkpoint. When delivered it would allow the patient to produce specific antibodies against the checkpoint as well as expressing the antigen that will complete with that checkpoint's ligand.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is an overview of a disclosed vaccine-induced dual mechanism PD1 vaccine.

FIG. 2 shows expression of anti-PD1ex IgG antibody in mouse serum after intramuscular delivery of PD1ex plasmid.

FIG. 3 Combination treatment with pIL-12 and pPD1P and pPD1N. Mice with established tumors were treated with one or two plasmids, 3 times in one week. N=10 for each group.

FIG. 4 Binding of PD1P to PDL1 on B16 tumors. B16.F10 tumors were treated with different GET conditions with or without heat. PD1+ cells present in tumors following dissociation. CD45 is a marker for hematopoietic cells. N=5 for each.

FIG. 5 Binding of PD1 peptides to PDL1 on B16.F10. Representative section from tumor treated with pPD1P. Green=Melan A+; yellow=PD1+; red=CD8+.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

“Impedance” as used herein refers to the opposition of an electric current to the flow of an alternating or direct current of a single frequency equal to the square root of the sum of the squares of the resistance and the reactance, expressed in ohms. Impedance may be measured at any frequency from 0 Hz to infinity. In some embodiments, impedance feedback is measured at any frequency below 4 kHz. In another embodiment, impedance feedback is measured between about 0 Hz to about 4 kHz.

“Pulse” or “pulsation” as used herein refers to a change in voltage or current intensity that lasts for a short duration of time. The duration of the pulses used herein last between about 1 μs to about 1 second. Examples of pulse polarity include unipolar and bipolar pulses. “4×4 pulsing” refers to two sets of four pulses being applied normal (90 degrees) to each other. For example, using 4 electrodes arranged in a square geometry, a first set of four pulses may be applied with electrodes 1 and 4 as positive and electrodes 2 and 3 negative. After a given time interval, a second set of four pulses in which electrodes 1 and 2 are positive and 3 and 4 are negative is applied. 4×4 pulsing can also be applied to multi-electrode arrays in which two sets of four pulses are applied in each sector in series. “2×2 pulsing” is similar except two pulses are applied in each direction. “Pulse number” as used herein refers to the number of pulses administered to the biological structure. The electric pulse may be rectangular, exponentially decaying, of any shape or combinations thereof. The pulse may be direct current, alternating current or combinations thereof.

“Electroporation” as used herein refers to the application of an electrical field to a biological structure, such as a cell or tissue, to increase the permeability of the cell membrane to allow molecules to be introduced to the cell.

“Electrotransfer” as used herein refers to the use of an electric field, such as through electroporation, to transfer molecules such as drugs or genetic material into cells, tissues, or other biological structures.

“Heating” or “applying heat” as used herein refers to the process in which the temperature of a biological structure is increased. Heating may be accomplished by any convective, conductive, or radiative means, including combinations thereof, known to those of skill in the art. Exemplary heating methods include, but are not limited to, application of warm air, contact with a warm surface, infrared radiation (IR), electromagnetic waves or emissions at any frequency, microwave emissions, chemical means such as chemical containing heat pads, and combinations thereof.

“Heating element” or “heat generation device” as used herein refers to any device capable of converting energy to heat. Exemplary heating elements include, but are not limited to, light emitting diodes (LEDs); chemical containing heat pads; electromagnetic wave generators; optic fibers connected to an infrared laser source; resistive heating elements composed of metallic alloys, ceramic materials or ceramic metals; and combinations thereof.

“Temperature measurement device” as used herein refers to any device capable of directly or indirectly measuring the temperature of a biological structure. Examples of temperature measurement devices include, but are not limited to, thermocouples, thermopiles, thermistors, infrared (IR) sensors, heat sensing cameras including infrared (IR) sensing cameras, impedance measurement devices, and combinations thereof.

“Temperature monitoring” as used herein refers to the process of directly or indirectly measuring the temperature of a biological structure over a period of time. Exemplary methods for temperature monitoring include, but are not limited to, impedance measurement; thermal imaging; temperature measurement devices such as thermistors, thermocouples, thermopiles, or any other temperature measurement device that directly or indirectly measures temperature or correlates temperature to a variable.

“Relay” as used herein refers to any device, switch, or means that can be used to address an electrode. Generally, the relay is activated by a current or signal in one circuit to open or close another circuit.

Disclosed herein are compositions and methods involving electrotransfer (ET) of molecules to tumor tissue. U.S. Pat. No. 10,814,129 is incorporated by reference in its entirety for the teaching of ET methods that use impedance-based monitoring at elevated temperatures to increase in vivo delivery by electroporation. As disclosed herein, these methods have the additional advantage of being able to control the amount and distribution of a molecule in tissues, such as tumor tissues.

In particular, disclosed herein is a method for bimodal immunotherapy in a subject that involves first delivering a first polynucleotide encoding a first molecule to a tumor tissue of the subject by a method that involves 1) applying heat to the tumor tissue to heat the tumor tissue to a preset temperature; applying at least one electroporation pulse to deliver the first polynucleotide into the tumor tissue; 2) measuring impedance of the tumor tissue as a feedback control mechanism after each pulse; and 3) adjusting pulse parameters based on the measured impedance of the tumor tissue until desired impedance is reached indicating delivery of the first polynucleotide to the tumor tissue;

The method can further involve delivering a second polynucleotide encoding a second molecule to a non-tumor tissue of the subject by a method that involves 1) applying heat to the tumor tissue to heat the non-tumor tissue to a preset temperature; applying at least one electroporation pulse to deliver the second polynucleotide into the tumor tissue; 2) measuring impedance of the non-tumor tissue as a feedback control mechanism after each pulse; and 3) adjusting pulse parameters based on the measured impedance of the tumor tissue until desired impedance is reached indicating delivery of the second polynucleotide to the non-tumor tissue.

In some aspects of the disclosed methods, the first polynucleotide is delivered to the tumor tissue in an effective amount to activate or maintain an immune response in the tumor tissue, and the second polynucleotide is delivered to the non-tumor tissue in an effective amount to activate an adaptive immune response in the subject.

In some examples, the first polynucleotide is delivered to the tumor tissue to activate or maintain an immune response in the tumor tissue. Suitably, the immune response is an anti-tumor immune response. Suitably, the immune response can be an adaptive immune response, for example, an antibody response, a cell-mediated immune response, or combination thereof. The anti-tumor immune response can be a T-cell response, such as a CD8+ T cell response or cytotoxic T lymphocyte (CTL response). Cellular immune responses are understood by one skilled in the art and include the ability to kill tumor cells. Activation of CD8+ T cells response leads to programmed cell death of tumor cells.

In some examples, the adaptive immune response is an antibody response or humoral immunity. Humoral immunity refers to antibody production and the coinciding processes that accompany it, including: Th2 activation and cytokine production, germinal center formation and isotype switching, and affinity maturation and memory cell generation. It also refers to the effector functions of antibodies, which include pathogen and toxin neutralization, classical complement activation, and opsonin promotion of phagocytosis and pathogen elimination. In some examples, the antibody response are anti-tumor antigen antibodies.

In another embodiment, the methods described herein elicit an antibody against the checkpoint molecules, allowing the antibodies to block the checkpoint molecules function to inhibit an immune response.

In some embodiments, the first polynucleotide encodes a checkpoint molecule. In some embodiments, the second polynucleotide encodes a checkpoint molecule. In some embodiments, both the first and second polynucleotide encode a checkpoint molecule.

In another embodiment, the first polynucleotide can encode a checkpoint molecule, a cytokine, a chemokine or combinations thereof, and the second polynucleotide encodes a checkpoint molecule, a cytokine or chemokine, or a combination thereof. In one example, the first polynucleotide can encode a checkpoint molecule and the second polynucleotide can encode a cytokine or chemokine.

The term “checkpoint molecule” used herein refers to a molecule that is expressed on a cell surface (e.g., tumor cells) and engages with proteins on the surface of immune cells. These immune checkpoint proteins when they bind their partner proteins, they send an “off” signal to the T cells to prevent the immune system from destroying the cell. In some embodiments, the checkpoint molecule comprises PD1, PDL1, CTLA-4, TIM-3, 4.1BB, LAG-3, CD80, CD86, OX40, OX40L, or a combination thereof.

In some examples, the cytokine or chemokine is selected from the group consisting of IL-12 (UniProtKB P29460 (IL12B_HUMAN)), IL-15 (UniProtKB P40933 (IL15_HUMAN), GM-CSF (UniProtKB P04141 CSF2_HUMAN, IFNA1 (UniProtKB P01562 (IFNA1_HUMAN)), IFNA2 (UniProtKB P01563 IFNA2_HUMAN)), IFNA4 (UniProtKB P05014 (IFNA4_HUMAN), IFNA5 (UniProtKB P05013 (IFNA6_HUMAN)), IFNA7 (UniProtKB P01567 (IFNA7_HUMAN), IFNA8 (UniProtKB P32881 (IFNA8_HUMAN)), IFN10 (UniProtKB P01566 (IFN10_HUMAN)), IFN14 (UniProtKB P01570 (IFN14_HUMAN)), IFN16 (UniProtKB P05015 (IFN16_HUMAN)), IFN17 (UniProtKB P01571 (IFN17_HUMAN)), IFN21 (UniProtKB P01568 (IFN21_HUMAN)), IFNB (UniProtKB P01568 (IFNB_HUMAN), IFNG (UniProtKB P01579 (IFNG_HUMAN)), IFNL1 (UniProtKB Q8IU54 (IFNL1_HUMAN)), IFNL2 (UniProtKB P05014 (IFNA4_HUMAN)), IFNL3 (UniProtKB Q81Z19 (IFNL3_HUMAN)), IFNL4 (UniProtKB K9M1U5 (IFNL4_HUMAN)), CCL19, (UniProtKB Q99731 (CCL19_HUMAN))CCL21 (UniProtKB O00585 (CCL21_HUMAN)), CCL14 (UniProtKB Q16627 (CCL14_HUMAN)), CXCL12 (SDF1) (UniProtKB P48061 (SDF1_HUMAN)), and CCR7 (UniProtKB P32248 (CCR7_HUMAN)), IL1A (UniProtKB P01583 (IL1A_HUMAN)), IL1B (UniProtKB P01583 (IL1A_HUMAN)), 1L2 (UniProtKB P60568 (IL2_HUMAN)), IL8 (UniProtKB P60568 (IL2_HUMAN)), CCL2 (UniProtKB P13500 (CCL2_HUMAN)), CCL3 (UniProtKB P10147 (CCL3_HUMAN)), CCL4 (UniProtKB P13236 (CCL4_HUMAN)), CCL5 (UniProtKB P13501 (CCL5_HUMAN)), CCL11 (UniProtKB P51671 (CCL11_HUMAN)), CXCL10 (UniProtKB P02778 (CXL10_HUMAN)).

An “effective treatment” refers to treatment producing a beneficial effect, e.g., amelioration of at least one symptom of a cancer. A beneficial effect can take the form of an improvement over baseline, i.e., an improvement over a measurement or observation made prior to initiation of therapy according to the method. A beneficial effect can also take the form of reducing, inhibiting or preventing further growth of cancer cells, reducing, inhibiting or preventing metastasis of the cancer cells or invasiveness of the cancer cells or metastasis or reducing, alleviating, inhibiting or preventing one or more symptoms of the cancer or metastasis thereof. Such effective treatment may, e.g., reduce patient pain, reduce the size or number of cancer cells, may reduce or prevent metastasis of a cancer cell, or may slow cancer or metastatic cell growth.

### Polynucleotides

In some embodiments, the first molecule is PD1 and the second molecule is PDL1.

For example, in some embodiments, the first polynucleotide encodes a PD1 polypeptide having the amino acid sequence of the nivolumab (Bristol-Myers Squibb) binding region: LDSPDRPWNP (SEQ ID NO:1, NP_005009.2). Therefore, in some embodiments, a polynucleotide encoding PD1 has the nucleic acid sequence: 5′TTAGACTCCCCAGACAGGCCCTGGAACCCC3′ (SEQ ID NO:2, NM_005018.3).

For example, in some embodiments, the first polynucleotide encodes a PD1 polypeptide having the amino acid sequence of the pembrolizumab (Merck) binding region: NQTDKLAAFPEDRSQPGQDCRFRVTQ (SEQ ID NO:3, NP_005009.2). Therefore, in some embodiments, a polynucleotide encoding PD1 has the nucleic acid sequence: 5′CAACCAGACGGACAAGCTGGCCGCCTTCCCCGAGGACCGCAGCCAGCCCGG CCAGGACTGCCGCTTCCGTGTCACACAA3′ (SEQ ID NO:4, NM_005018.3).

In some embodiments, the first polynucleotide encodes a peptide comprising, e.g., CTLA4 (UniProtKB P16410 (CTLA4_HUMAN)), TIM3 (HAVR2 UniProtKB (Q8TDQ0 (HAVR2_HUMAN)), 4-1BB (TNR9, UniProtKB Q07011 (TNR9_HUMAN)), LAG3 (UniProt KB P18627 (LAG3_HUMAN)), CD80 (UniProtKB-P33681 (CD80_HUMAN)), CD86 (UniProtKB-P42081 (CD86_HUMAN)), OX40 (UniProtKB-P43489 (TNR4_HUMAN)), OX40L UniProtKB-P23510 (TNFL4_HUMAN)), or a portion of thereof.

The polynucleotides of the instant disclosure are expressed by one or more nucleic acid constructs in the donor cell. The term “nucleic acid construct,” “construct” and “expression construct” refer to a polynucleotide sequence encoding the protein of interest and a promoter operably connected to a polynucleotide. The polynucleotide sequence may comprise heterologous backbone sequence. The nucleic acid sequence may be a vector. As used herein, the term “vector” refers to a nucleic acid molecule capable of propagating another nucleic acid to which it is linked. Suitable vectors for use with the present invention comprise a promoter operably connected to a polynucleotide sequence encoding the fusion peptide described herein. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors” (or simply, “vectors”). The term vector encompasses “plasmids”, the most commonly used form of vector. Plasmids are circular double-stranded DNA loops into which additional DNA segments (e.g., encoding a first and/or second polynucleotide) may be ligated. Certain vectors are capable of autonomous replication in a host cell into which they are introduced (e.g., bacterial vectors having a bacterial origin of replication and episomal mammalian vectors). Other vectors may be integrated into the genome of a host cell upon introduction into the host cell and are thereby replicated along with the host genome. The vectors may also comprise appropriate control sequences that allow for translational regulation in a host cell. In some embodiments, the vectors further comprise the nucleic acid sequences for one or more additional proteins. In some embodiments, the vectors further comprise additional regulatory sequences, such as signal sequences.

In some embodiments, the vectors of the present invention further comprise heterologous backbone sequence. As used herein, “heterologous nucleic acid sequence” refers to a non-human nucleic acid sequence, for example, a bacterial, viral, or other non-human nucleic acid sequence that is not naturally found in a human. Heterologous backbone sequences may be necessary for propagation of the vector and/or expression of the encoded peptide. Many commonly used expression vectors and plasmids contain non-human nucleic acid sequences, including, for example, CMV promoters.

### Electroporation Methods

Systems and methods for targeted delivery of molecules using impedance-based monitoring at elevated temperatures are described in U.S. Pat. No. 10,814,129, which is incorporated by reference in its entirety for these teachings.

In the prior procedure, a fixed set of pulses, having fixed pulse parameters, were applied at ambient tissue temperature with no attempt being made to control the temperature or customize pulsation. In contrast, in the impedance-based monitoring at elevated temperatures, the temperature of the local tissue (skin) area is increased and maintained at a constant preset temperature with impedance spectroscopy being used to measure the resulting tissue condition after every electroporation pulse. Adjustments can be made after measurement of the tissue condition by applying an additional pulse or stopping pulsing. Two additional physical parameters that markedly increase the success of in vivo gene delivery by electroporation. It was found that modest localized temperature increases in skin (43° C.) during DNA delivery resulted in an increase in expression. Further, the temperature increases allowed the magnitude of the applied pulses (voltage/field intensity) to be reduced by about 50% to achieve the same expression when compared to optimal delivery performed at ambient temperature. Similarly, adjusting pulse parameters during electrical treatment based upon real-time tissue impedance measurements resulted in between 6- to 15-fold increases in expression. It was found that pulse magnitudes can be reduced by 50% and still achieve increased expression relative to traditionally optimized conditions. The benefits of manipulating either physical parameter are compelling on their own. However, the combination of localized temperature increases and impedance-based feedback pulsing exhibit at least additive, if not synergistic, effects. The combination treatment provides better control, reduces variation, and further reduces the magnitude of pulses required for delivery.

Currently, delivery of molecules via electrotransfer is done by predetermining the number of pulses, pulse width and amplitude and then using that as a fixed set of parameters for each animal or patient treated. The problem with this approach is that each individual has different tissue properties even if the location between individuals is similar. This is particularly true with respect to the conductance of the tissue and the relative temperature. In addition, within a particular tissue there may also be areas of higher conductance. Therefore, using a standardized approach to pulsing would result in high variability from patient to patient and would also cause uneven distribution of delivery within the tissue.

Controlling process based upon the two physical parameters of temperature and impedance can reduce or virtually eliminate this variability with the tissue and between subjects thus increasing delivery and reproducibility of electroporation-based drug/gene delivery methods thus moving gene therapy closer to recombinant protein drug therapy. In addition, by monitoring both temperature and impedance, one can target the delivery to specific areas within the tissue. Enhancing tissue targeting and controlling dosing by controlling the amount and site of delivery also increases safety and reliability. While the method is described herein as being used on the skin, the method is applicable to any tissue or abnormal growth through the use of catheters, scopes or surgery.

The preset temperature may be at least 35° C. or more specifically, between about 40° C. to about 46° C. In some embodiments, the temperature can be selected from the group consisting of 35° C., 36° C., 37° C., 38° C., 39° C., 40° C., 41° C., 42° C., 43° C., 44° C., 45° C. and 46° C., including all intervening temperatures. The heat applied to the tissue may be transferred to the tissue by means of a convection, conduction, radiation or combinations thereof.

The pulse parameters may be selected from the group consisting of electric field intensity, pulse duration, pulse polarity, time interval between pulses, and number of pulses administered to the tissue (pulse number). The electric field intensity may be between about 5 V/cm to about 2000 V/cm, including all intervening values. The pulse duration may be between about 1 μs to about 1 second, including all intervening values. The time interval between pulses may be between about 1 μs to about 1 second, including all intervening values. The desired impedance may be at least 10% reduction in impedance as compared to pre-pulse impedance. The impedance feedback may be measured in a range of frequencies from 0 Hz to infinity, preferably between 0 Hz to 4 kHz.

The system for the delivery of a molecule into a tissue comprising: an electroporation device; an electric field generator used to apply pulses to a tissue and coupled to the at least one relay; an impedance measurement system coupled to the at least one relay; and a controller coupled to the at least one relay. The electroporation device is comprised of a handle having proximal and distal ends; an electrode array comprising a plurality of individually addressable electrodes attached at the distal end of the handle; at least one relay for addressing each electrode individually or in combination; at least one heating element disposed within the handle positioned proximal to the electrode array; and a temperature measurement system positioned to measure the temperature of the tissue.

The at least one heating element may be at least one light emitting diode (LED). The at least one heating element may be at least one resistive heating element. The temperature measurement system may be an infrared sensing camera. The impedance measurement system may be a low voltage impedance spectroscope.

The hardware required for temperature control and impedance measurement is capable of being adapted to current electroporation systems as such systems all have electrodes that are in contact with the target tissue during treatment. While electrode arrangement can differ between devices, current devices can be adapted to a preferred electrode arrangement to allow for temperature increases and impedance feedback. For example, an electrode arrangement of four electrodes may be used in some applications. In some embodiments, a multi-electrode array (MEA) may be used which may be comprised of nine subsets of four electrodes with each set of four electrodes comprising a sector within the overall array. In some embodiments, an optical fiber located within each sector for infrared emission to provide focused tissue heating. In other embodiments, heating elements and temperature measurement devices are disposed within the electroporation device. While these electrode arrangements are exemplary, any arrangement that allows for temperature increases and impedance feedback may be used. Alternatively, the heating and control systems may be added to existing electrode and pulse generators with some adaptation using electrically actuated switches or relays.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1

Disclosed herein is a simple option that does not require extensive plasmid engineering, a dual-mechanism PD1 vaccine. The PD-1 protein contains cytoplasmic, transmembrane, and extracellular domains. We have designed plasmids focusing on the extracellular domain or subdomains of PD1 to act as PD1 vaccines. Each plasmid includes an IgK secretion sequence in frame with mouse PD1 sequence to produce a soluble protein. The first plasmid, pPD1ex, encodes the entire mouse PD-1 extracellular region (amino acids 21-169). The second plasmid, pPD1N, encodes the mouse sequence homologous to nivolumab (SEQ ID NO: 1, Opdivo, Bristol-Myers Squibb) binding region. Finally, the third plasmid, pPD1P, encodes the mouse sequence homologous to the pembrolizumab (SEQ ID NO: 3, Keytruda, Merck) binding region. While the current constructs are for mouse PD1, the same concepts can be applied to human PD1. Sequences are similar (mouse sequences are designed based the human sequences) and are well described and available.

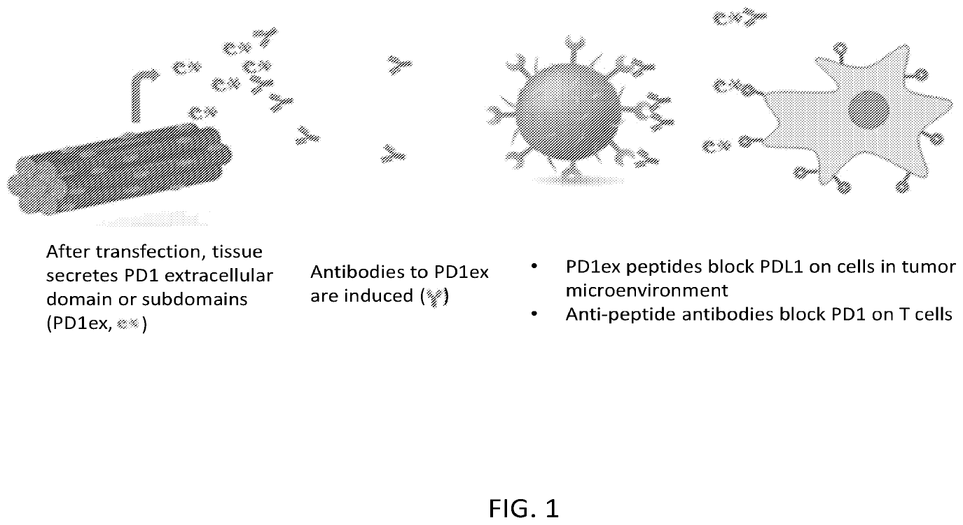

The proposed anti-PD1 gene therapy works by dual mechanisms; each mechanism may be effective individually. The plasmids encode soluble peptides of PD1, which may bind PDL1 on tumor cells to block normal PD1-PDL1 binding (FIG. 1). In parallel, this peptide will act as an antigen to induce systemic and polyclonal checkpoint inhibitor antibodies forming an independent blockade to PD1-PDL1 binding. These polyclonal antibodies target the same region as the monoclonal antibodies and should function similarly to monoclonal checkpoint inhibitor antibodies. In some cases, the PD1 antigen and antibody will bind each other, which would result in a dead-end response. This multipurpose mechanism should allow the proliferation of T cells in the tumor microenvironment while limiting the need for multiple monoclonal antibody injections.

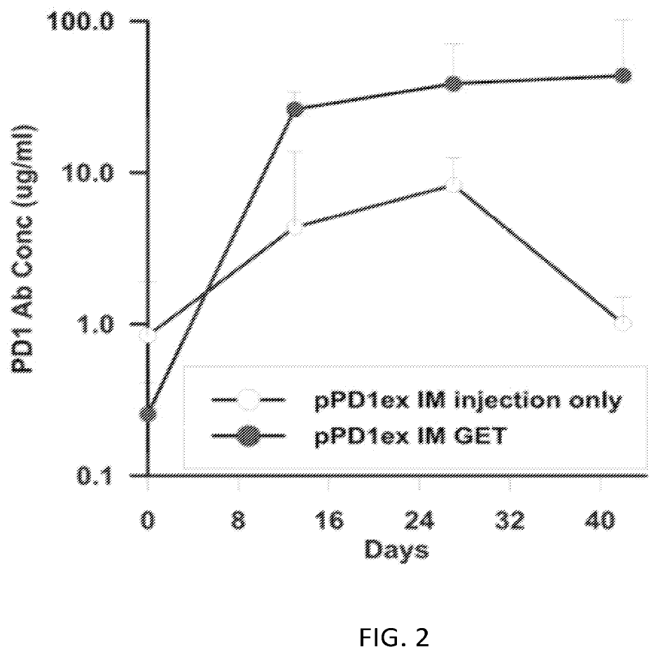

IM electroporation of the PD1ex plasmid in mice induces significant, long-term production of anti-PD1 antibody in serum (FIG. 2).

Unless defined otherwise, all technical and scientific terms used herein have the same meanings as commonly understood by one of skill in the art to which the disclosed invention belongs. Publications cited herein and the materials for which they are cited are specifically incorporated by reference. Those skilled in the art will recognize, or be able to ascertain using no more than routine experimentation, many equivalents to the specific embodiments of the invention described herein. Such equivalents are intended to be encompassed by the following claims.

### Example 2

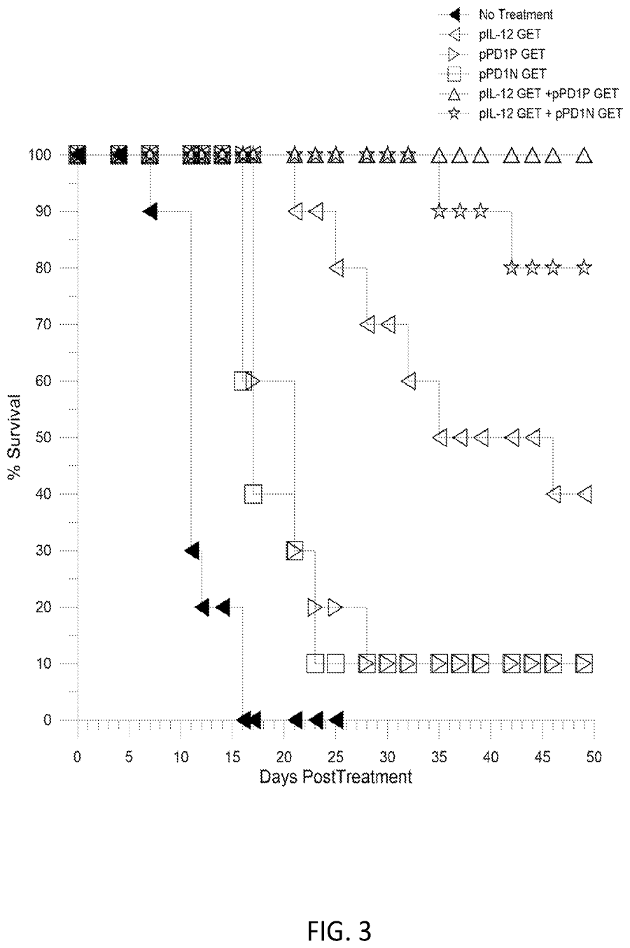

Experiments were performed to evaluate the anti-tumor effect of combining intratumor delivery of plasmid encoding PD1 peptides (pPD1P and pPD1N) either alone or in combination with plasmid encoding IL-12 (pIL-12) all of these plasmids were delivered with gene electrotransfer (GET). Tumors were established by injecting B16.F10 cells (1×10⁶) into the left flank of C57Bl/6 mice. Delivery was performed when tumors were approximately 50 mm³. GET conditions utilized to deliver the plasmids were 600 V/cm 5 ms pulse width and 10 pulses. IL-12 plasmid was delivered on Days 0, 4 and 7 and when used in combination with pIL-12, pPD1P and pPD1N were delivered on Days 1, 5 and 8. The highest survival and best tumor response were seen when combining pIL-12 GET with pPD1P or pPD1N (FIG. 3).

An important aspect related to delivery of pPD1P and pPD1N directly to the tumor is to have the expressed peptide bind to PDL1 on tumor cells. To test the feasibility of this concept, B16.F10 tumors were established in the left flank. Delivery was performed when tumors were approximately 50 mm³. The two plasmids were injected with 100 μg of plasmid without GET or delivered with GET using a field strength of 600 V/cm at 5 ms pulse width and 10 pulses; or 600 V/cm at 5 ms pulse width and 10 pulses with heat or 150 V/cm at 150 ms pulse width and 10 pulses with heat. Two days after delivery, mice were humanely euthanized and tumors removed. Half of the tumors were evaluated by flow cytometry and half were evaluated by immunohistochemistry (IHC)*.

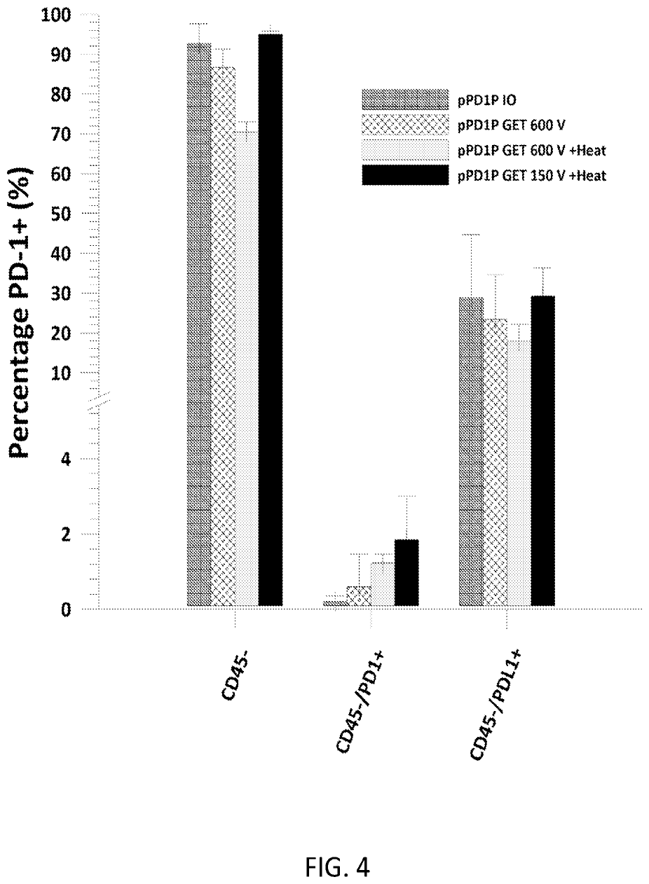

Tumor cells do not express CD45⁻ or PD1, but do express PDL1. So, detecting PD1 peptide on CD45⁻ cells suggests that pPD1P is expressed and secreted and in turn is binding to PDL1 on the surface of these cells. Flow cytometry revealed that the use of GET increases production and subsequent binding of PD1 to CD45⁻ cells. It is also clear that GET+heat further increases expression and binding. (FIG. 4)

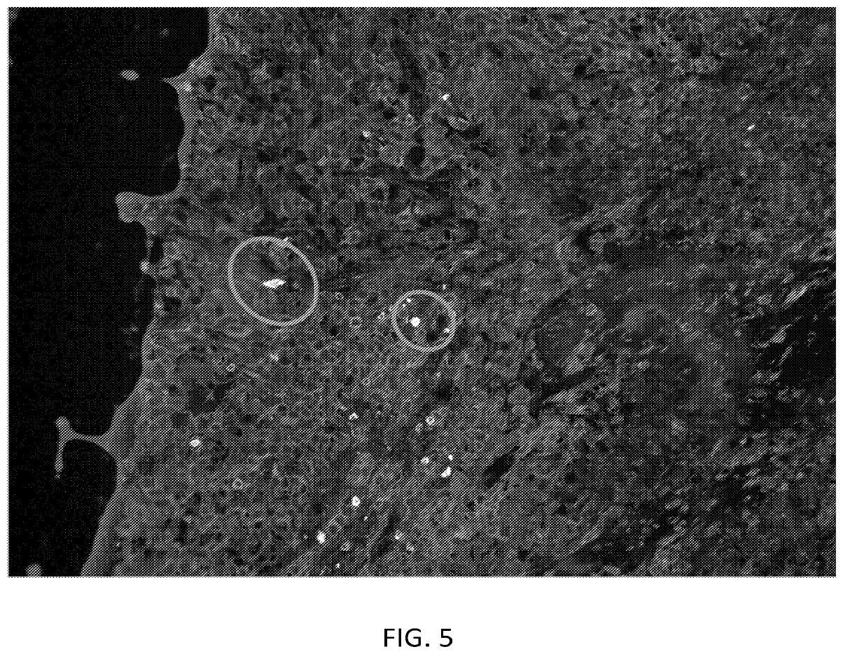

Tumors were stained by IHC for the presence of MelanA (marker on B16 cells) and PD1. Following delivery of the two plasmids there are clearly dual stained cells (FIG. 5). While levels of binding were not high, this was evaluated after a single treatment and a single dose. It does, however, show that binding can be achieved.

1. A method for bimodal immunotherapy in a subject, comprising
(a) delivering a first polynucleotide to a tumor tissue of the subject by a method that comprises:
(1) applying heat to the tumor tissue to heat the tumor tissue to a preset temperature; applying at least one electroporation pulse to deliver the first polynucleotide into the tumor tissue,
(2) measuring impedance of the tumor tissue as a feedback control mechanism after each pulse, and
(3) adjusting pulse parameters based on the measured impedance of the tumor tissue until desired impedance is reached indicating delivery of the first polynucleotide to the tumor tissue; and
(b) delivering a second polynucleotide to a non-tumor tissue of the subject by a method that comprises:
(1) applying heat to the non-tumor tissue to a preset temperature; applying at least one electroporation pulse to deliver the second polynucleotide into the tumor tissue,
(2) measuring impedance of the non-tumor tissue as a feedback control mechanism after each pulse, and
(3) adjusting pulse parameters based on the measured impedance of the tumor tissue until desired impedance is reached indicating delivery of the second polynucleotide to the non-tumor tissue,
wherein the first polynucleotide is delivered to the tumor tissue in an effective amount to activate or maintain an immune response in the tumor tissue, and
wherein the second polynucleotide is delivered to the non-tumor tissue in an effective amount to activate an adaptive immune response in the subject.

2. The method of claim 1, wherein the first polynucleotide encodes a checkpoint molecule, wherein the second polynucleotide encodes a checkpoint molecule, or a combination thereof.

3. The method of claim 2, wherein the checkpoint molecule comprises PD1, PDL1, CTLA-4, TIM-3, 4.1BB, LAG-3, CD80, CD86, OX40, OX40L, or a combination thereof.

4. The method of claim 3, wherein the first polynucleotide encodes PD1 and the second polynucleotide encodes PDL1.

5. The method of claim 3, wherein the first polynucleotide encodes is PD1 and the second polynucleotide encodes PD1.

6. The method of claim 1, wherein the first polynucleotide encodes a cytokine or chemokine, wherein the second polynucleotide encodes a cytokine or chemokine, or a combination thereof.

7. The method of claim 6, wherein the cytokine or chemokine is selected from the group consisting of IL-12, IL-15, GM-CSF, IFNs, CCI19, CCL21, CXCL12, CCL14, and CCR7.

8. The method of claim 1, wherein step (a) and step (b) are conducted within 1 hour of each other.

9. The method of claim 1, wherein step (a) and step (b) are conducted within 4 days of each other.

10. The method of claim 1, wherein step (a) and/or step (b) is repeated on a different day.

11. The method of claim 1, wherein the desired impedance is an at least a 10% reduction as compared to the measured impedance prior to each electroporation pulse.

12. The method of claim 1, further comprising monitoring temperature of the tumor tissue, non-tumor tissue, or a combination thereof.

13. The method of claim 12, wherein the temperature is monitored using impedance, thermal imaging, thermistors, thermocouples, thermopiles or combinations thereof.

14. The method of claim 1, wherein the preset temperature is at least 35° C.

15. The method of claim 14, wherein the preset temperature is from 40° C. to 46° C.

16. The method of claim 1, wherein the heat applied to the biological structure is convective, conductive, radiative or combinations thereof.

17. The method of claim 1, wherein the impedance feedback is measured in a frequency range of from 0 Hz to 4 kHz.

18. The method of claim 1, wherein the pulse parameters are selected from the group consisting of electric field intensity, pulse duration, pulse polarity, time interval between pulses, and number of applied pulses.

19. The method of claim 18, wherein the electric field intensity is from 5 V/cm to 2000 V/cm.

20. The method of claim 18, wherein the pulse duration is from 1 μs to 1 second or wherein the time interval between pulses is from 1 μs to 1 second.

21. (canceled)
